(19) United States
(12) Patent Application Publication
(10) Pub. No.: US 2013/0023782 A1
(43) Pub. Date: Jan. 24, 2013

(54) GAS SENSOR, ANALYZER AND METHOD FOR MEASURING OXYGEN CONCENTRATION OF A RESPIRATORY GAS

(71) Applicant: Kai Karlsson, Helsinki (FI)

(72) Inventor: Kai Karlsson, Helsinki (FI)

(73) Assignee: GENERAL ELECTRIC COMPANY, Schenectady, NY (US)

(21) Appl. No.: 13/184,820

(22) Filed: Jul. 18, 2011

(51) Int. Cl. A61B 5/08

(52) U.S. Cl. 600/532

(57) ABSTRACT

A gas sensor is disclosed herein. The gas sensor includes an emitter for emitting radiation to a body at least partly coated with a luminophore emitting luminescent radiation indicative of an oxygen concentration when in contact with a respiratory gas. The gas sensor also includes a filter for transmitting the luminescent radiation emitted by the luminophore and an oxygen detector for receiving the luminescent radiation transmitted by the filter. The gas sensor also includes an infrared thermometry unit for receiving a thermal radiation from the luminophore. A gas analyzer and a method for measuring oxygen concentration of a respiratory gas are also provided.

### BACKGROUND OF THE INVENTION

This disclosure relates generally to a gas sensor including an emitter for emitting radiation to a body at least partly coated with a luminophore emitting luminescent radiation indicative of an oxygen concentration when in contact with a respiratory gas, a filter for transmitting the luminescent radiation emitted by the luminophore and an oxygen detector for receiving the luminescent radiation. This disclosure also relates to a gas analyzer and method for measuring oxygen concentration of a respiratory gas

In anesthesia or in intensive care, the status of a patient is often monitored by analyzing the gas inhaled and exhaled by the patient for its content. For this reason either a small portion of the respiratory gas is diverted to a gas analyzer or the gas analyzer is directly connected to the respiratory circuit. The former analyzer is of sidestream type, the latter is named mainstream because of its ability to measure directly across the respiratory tube. Typical for the mainstream sensor is that it has a disposable airway adapter and a directly connectable sensor body. The majority of the mainstream sensors on the market are designed to measure carbon dioxide alone, using infrared non-dispersive (NDIR) absorption technique. The basis of this technique is well-known and is explained in detail in literature and patents. As it is not directly related to this case the NDIR measurement will not be further described in this document.

Another gas of vital importance is, of course, oxygen. Oxygen can be measured using chemical sensors or fuel cells, but they are normally too bulky to fit into a mainstream sensor and, although they have a limited lifetime, they are not designed to be single use and must therefore be protected from direct contact with the patient gas to avoid contamination. This is expensive and also influences the response time of the sensor. Oxygen can also be measured using a laser and the absorption at 760 nm. However, this absorption is very weak and the signal from the short distance across the respiratory tube becomes too noisy to be useful. The most promising method is luminescence quenching. A special sensor coating, a luminophore, is excited using e.g. blue light from a light emitting diode (LED). A luminescence signal can be detected at longer wavelengths, often in the red portion of the spectrum. Oxygen has the ability to quench this luminescence in a predictable way by consuming the available energy directly from the luminophore. Thus, the amount of quenching is a direct measure of the partial pressure of oxygen in the respiratory gas mixture. Luminescence quenching offers the possibility to make a single use probe in connection with the patient adapter. Problems that have to be attended to are temperature and humidity dependence as well as drift caused by ageing. Normally the luminescence intensity is not measured directly but a change in the decay time of the excited state is a more stable and robust measurable. Still, an optical reference is normally a necessity as is also temperature compensation.

In the clinically used gas analyzer of mainstream type the whole volume or at least the main portion of the breathing air or gas mixture flows through the analyzer and its disposable measuring chamber. Because the measuring chamber is in the breathing circuit, it is easily contaminated by mucus or condensed water. Thus, it is necessary to use sensors that are as robust and insensitive to the difficult conditions as possible. The infrared sensor uses one or more reference wavelengths in a mainstream analyzer in order to have good enough estimate of the signal level without gas absorption, the zero level, continuously available. For the oxygen sensor it is important that contamination does not alter the sensitivity more than what can be tolerated. The sensor based on luminescence quenching seems to fulfill this demand. It is known that it works also submerged in water as it measures the dissolved oxygen. The response time will naturally be longer in such a measurement.

A clinical mainstream gas analyzer must be small, light, accurate, robust and reliable. The analyzer must maintain its accuracy in widely varying operating conditions. For example, many clinical gas analyzers are specified to operate at ambient temperatures between +10 and +35 C, and the tubes conducting breathing gases may be at ambient temperature or kept at a known temperature to avoid water condensation. Also, the temperature of the luminophore is affected by the flowing gas in contact with the luminophore. In clinical use, the temperature of expiration gas will be close to the patient's body temperature and the temperature of the inspired gas will be close to the temperature of the inspiration tube from the ventilator to the patient. It is not possible make a zeroing measurement using a reference gas during normal operation. Because the luminescent properties of luminophores depend on temperature, the luminophore must either be kept at a known temperature or its temperature must be measured and taken into account in the calculation of partial pressure of oxygen. The latter method is very much preferred because of the bulkiness and power consumption of thermostat heating or cooling systems.

Yet, the analyzer must maintain its accuracy even if the measuring chamber would be contaminated. Due to these requirements, mostly single gas mainstream analyzers for carbon dioxide (CO₂) have been commercially available. A really compact CO₂ and O₂ gas analyzer has been technically very challenging.

Another requirement is that the measurement has to be fast enough to measure the breathing curve. In practice, the rise time would have to be in the order of 200 ms or even shorter. For CO₂ this is possible to arrange using well known infrared measuring technique. The luminescent O₂ sensor must have a very thin layer of active material in order to react fast enough. This decreases the signal and to compensate for that the sensor surface must be increased.

Oxygen sensors of prior art based on luminescence quenching in a mainstream adapter include a window that transmits the radiation involved to and from a surface coated with a luminophore. The window may be very thin so that the window can be a membrane. The measurement method is well known and it is also known that a sensor can be kept at 37+/−0.1 C temperature and has an additional microchip thermistor for measuring the instantaneous temperature of the fluorophore. This kind of thermistor is fastened to the window coated with the luminophore, but unfortunately is not able to follow constantly changing temperature fast enough as is the case with the respiratory measurement. Also the main stream adapter with the thermistor fastened to the window is too expensive to be disposable and should therefore be sterilized after each use.

### BRIEF DESCRIPTION OF THE INVENTION

The above-mentioned shortcomings, disadvantages and problems are addressed herein which will be understood by reading and understanding the following specification.

In an embodiment a gas sensor includes an emitter for emitting radiation to a body at least partly coated with a luminophore emitting luminescent radiation indicative of an oxygen concentration when in contact with a respiratory gas and a filter for transmitting the luminescent radiation emitted by the luminophore. The gas sensor also includes an oxygen detector for receiving the luminescent radiation transmitted by the filter and an infrared thermometry unit for receiving a thermal radiation indicative of a temperature of the luminophore.

In another embodiment a gas analyzer for measuring oxygen concentration of a respiratory gas includes an emitter for emitting radiation and an airway adapter having a flow channel carrying respiratory gas including oxygen. The gas analyzer also includes a body at least partly coated with a luminophore excited by the radiation emitted by the emitter, the luminophore being in contact with the respiratory gas and emitting luminescent radiation. The gas analyzer further includes a filter for transmitting the luminescent radiation emitted by the luminophore and an oxygen detector for receiving the luminescent radiation transmitted by the filter. The gas analyzer also includes an infrared thermometry unit for receiving a thermal radiation from the luminophore.

In yet another embodiment a method for measuring oxygen concentration of a respiratory gas includes emitting a radiation to a body coated at least partly with a luminophore which luminophore is adapted to emit luminescent radiation indicative of an oxygen concentration when in contact with the respiratory gas and filtering the radiation to transmit the luminescent radiation. The method also includes detecting the transmitted luminescent radiation and receiving a thermal radiation from the luminophore indicatice of a temperature of the luminophore.

Various other features, objects, and advantages of the invention will be made apparent to those skilled in art from the accompanying drawings and detailed description thereof.

### BRIEF DESCRIPTION OF THE DRAWINGS

FIG. 1 illustrates a medical mainstream gas analyzer connected to the ventilation circuit of a patient.

FIG. 2 shows a gas analyzer comprising an airway adapter and a gas sensor including an oxygen measuring principle in accordance with an embodiment;

FIG. 3 shows an oxygen measuring principle and components in accordance with the another embodiment;

FIG. 4 shows an oxygen measuring principle and components in accordance with another embodiment;

FIG. 5 shows an oxygen measuring principle and components in accordance with another embodiment; and

FIG. 6 shows an oxygen measuring principle and components in accordance with another embodiment.

### DETAILED DESCRIPTION OF THE INVENTION

Specific embodiments are explained in the following detailed description making a reference to accompanying drawings. These detailed embodiments can naturally be modified and should not limit the scope of the invention as set forth in the claims.

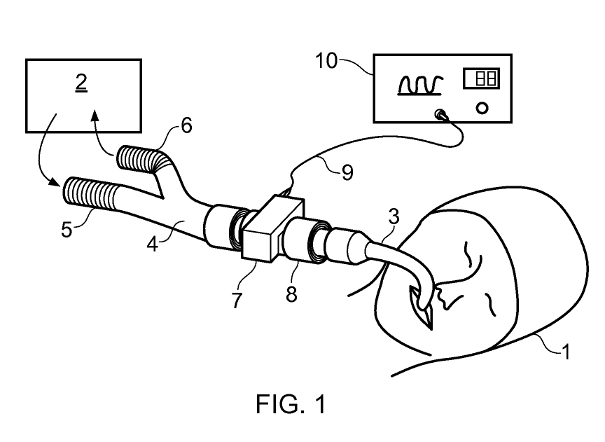

A gas analyzer 7 for measuring a respiratory gas such as oxygen is shown in FIG. 1. This technology may be applied in clinical multigas analyzers of mainstream type. The gas analyzer 7 such as a medical mainstream gas analyzer may be measuring directly across the respiratory tube of an intubated patient 1 as shown in FIG. 1. The patient 1 is connected to a ventilator 2 using an intubation tube 3, a Y-piece 4, an inspiratory limb 5 and an expiratory limb 6. The airway adapter 8 is connected to the intubation tube. The gas analyzer 7 which comprises components of the airway adapter is electrically connected via cable 9 to the patient monitor 10. The gases measured may be besides oxygen O₂ also carbon dioxide CO₂ and possibly other gases with infrared absorption like nitrous oxide N₂O and anesthetic gases.

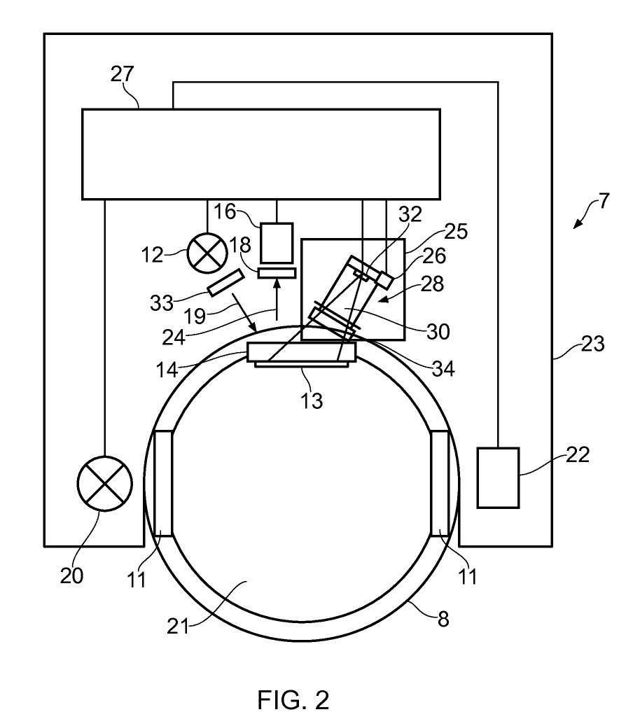

In FIG. 2 a close-up of the gas analyzer 7 comprising a gas sensor 23 and the airway adapter 8 is depicted. The gas sensor 23 may be mountable on the airway adapter 8. The airway adapter 8 normally may be disposable. This adapter may be provided with two infrared transmitting windows 11, which are needed in case other respiratory gases than oxygen are measured. An infrared source 20 is located in the gas sensor 23 emitting radiation through the windows 11 having therebetween a flow channel 21 for the respiratory gas flowing between the patient and the ventilator 2. At least one gas detector 22 for providing a signal indicative of at least one respiratory gas other than oxygen is needed and which gas detector is located also in the gas sensor so that it is on another side of the adapter than the infrared source. Typically also a non-dispersive filter assembly (not shown in Figure) is between the infrared source 20 and the gas detector 22. Thus the infrared radiation is directed from the infrared source through the windows 11 and respective narrowband filters to the gas detector or detectors 22. The signal from each detector is amplified and modified to reflect the concentration of the gas to be measured or it may be a measurement at a reference wavelength with no or little gas absorption. As mentioned above, respiratory gases can be carbon dioxide, nitrous oxide and different volatile anesthetic agents. All these gases absorb infrared radiation within some specific wavelength region and this region is selected using narrowband filters. The NDIR gas measuring technique is well known and will not be further described here. Gases like oxygen that do not absorb enough infrared radiation using the short measuring channel between the windows 11, can be measured using a different principle based on luminescence quenching because of a number of additional benefits.

According to an embodiment shown in FIG. 2 the gas sensor of the gas analyzer 7 for measuring oxygen concentration of the respiratory gas comprises an emitter 12 for emitting radiation. Especially the airway adapter 8 or alternatively the gas analyzer or the gas sensor 23 comprises a body 14, such as a window at least partly coated with a luminophore 13 exited by a radiation emitted by the emitter 12 and which luminophore is emitting luminescent radiation indicative of oxygen concentration of the respiratory gas when the luminophore is in direct contact with the respiratory gas. The luminophore can be a membrane on the surface of the body. The body 14 can be made of a transparent polymer and is therefore inexpensive. Of course, it could also be made of glass or any other transparent solid material like ceramic. The body 14 is advantageously rigid comprising a transparent radiation path for the radiation exciting the luminophore, the luminescent radiation emitted by the luminophore and the infrared radiation thermally emitted by the luminophore. The gas sensor 23 also comprises a filter 18 for transmitting luminescent radiation emitted by the luminophore 13 and an oxygen detector 16 for receiving the luminescent radiation transmitted by the filter 18. The optical filter 18 in front of the detector 16 is normally needed to filter out the radiation including light wavelengths from the emitter 12 and also disturbing ambient light, if such exists, transmitting only luminescence radiation, which normally has its maximum in the red end of the spectrum. The oxygen detector may provide a signal based on the received luminescent radiation indicative of an oxygen concentration.

The gas sensor 23 may be provided with specific arrangements to transmit the exciting radiation from the emitter 12, such as a light emitting diode (LED), and to reflect the luminescence radiation such as the light emitted by the luminophore 13 to the oxygen detector 16. The LED according to well-known technique often emits in the blue region but also yellow light has been used as exciting radiation, depending on the chemical composition of the luminophore. The emitter 12 may be equipped with an optical filter 33 to remove the possible infrared part of its emission.

The gas analyzer 7 according to this embodiment also includes an infrared thermometry unit (25) for receiving a thermal radiation from the body 14 whose surface is coated with the luminophore 13 being indicative of the temperature of the luminophore. The body may be advantageously so thin that the temperatures of its opposite surfaces are close enough to each other in case one of those opposite surfaces is the one coated with the luminophore. Also it is possible to make a body of a material such as calsiumflouride penetrating infrared radiation in which case the thickness of the body is not so critical. The infrared thermometry unit 25 comprising an infrared detector 32 for receiving the thermal radiation may provide a signal based on the received thermal radiation indicative of a temperature of the luminophore 13.

Further the infrared thermometry unit 25 may comprise in front of the infrared detector 32 an optical system 28 to limit the field of view of the infrared detector 25 to a suitable portion of the luminophore (13) and collecting radiation thermally emitted from that portion to the infrared detector. To achieve this the optical system may comprise an optical filter 34 for passing a suitable range of IR wavelengths to the infrared detector 25, an aperture 30 for limiting the field of view of the infrared detector 32 and temperature sensor 26 for measuring the temperature of the infrared detector. The temperature sensor 26 may provide a signal based on the temperature of the infrared detector 32. The infrared thermometry unit 25 and the infrared detector 32 is detached from the body 14 and at a distance from this body making possible to place the body with the luminophore 13 in the airway adapter 8 which may be detachable and disposable. The infrared detector 32 can instead locate outside the airway adapter 8 in the gas sensor 23. Expensive components are in the gas sensor, which is reusable and less expensive components are in the airway adapter 8 which is disposable to prevent contaminations between patients.

In case the infrared detector 32 may be placed closer to the body 14 than in FIG. 2 the optical system 28 can be omitted in front of the infrared detector 32 between the emitter or actually the luminophore and the infrared detector. When the infrared detector is close enough to the luminophore but however apart or at a distance from it, the infrared detector 32 is able to collect only the infrared radiation from the luminophore avoiding from collecting other radiation from an environment.

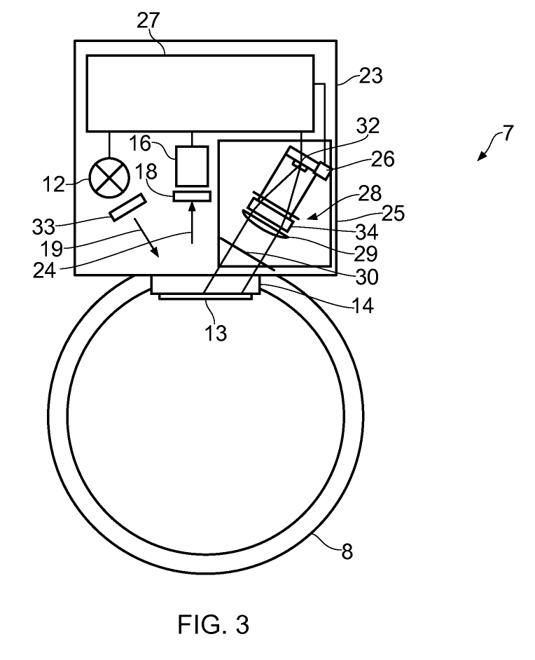

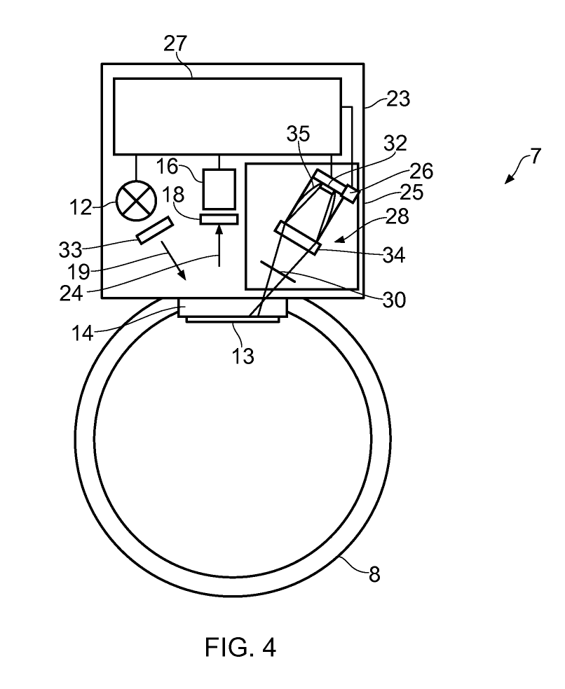

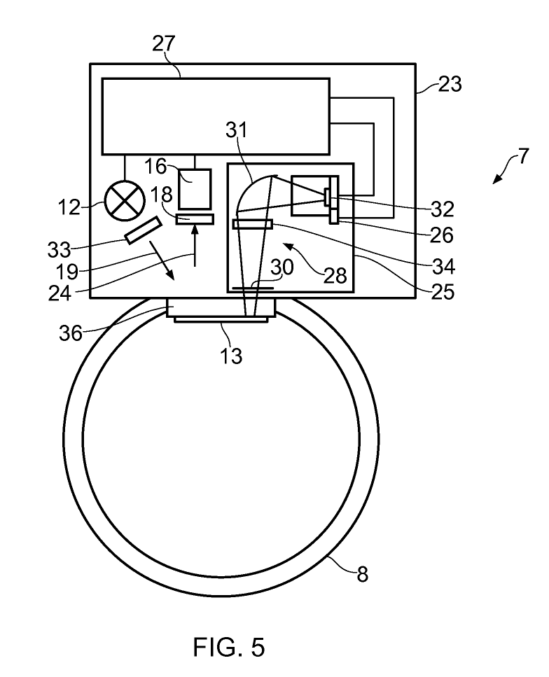

Other embodiments for measuring the temperature of the luminophore 13 are show in FIGS. 3, 4, 5 and 6. In FIG. 3 the optical system 28 comprises a lens 29 for collecting and focusing the thermal radiation and an aperture 30 for limiting the field of view of the infrared detector 32. In FIG. 4 the optical system 28 comprises a reflector 35 reflecting the thermal radiation passed through the aperture 30 for limiting the field of view and the optical filter 34 as disclosed hereinabove. The optical system 28 for limiting the field of view of the infrared detector in FIG. 5 comprises a mirror 31, the optical filter 34 and an aperture 30. The mirror is reflecting the thermal radiation passed though the aperture and the optical filter to the infrared detector 32 Otherwise the gas sensor in FIGS. 4 and 5 is similar to the one shown in FIGS. 2 and 3.

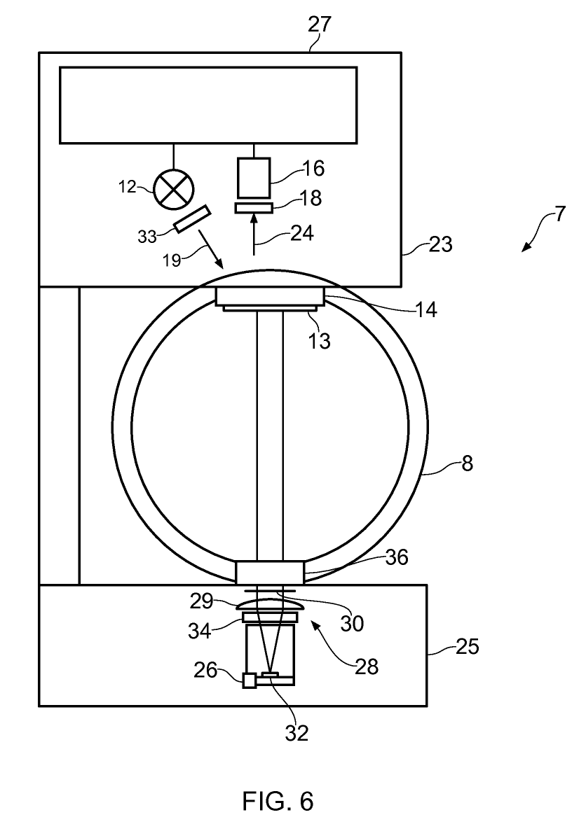

In FIG. 6 the design of the gas analyzer 7 differs from the ones introduced hereinbefore, because the infrared thermometry unit 25 locates opposite the flow channel 21 and the luminophore 13. The infrared thermometry unit can also locate anywhere around the airway adapter 8 looking towards the luminophore 13. The structure of the gas sensor is similar than the one shown in FIG. 3, where the lens 29, optical filter 34 and the aperture 30 formed the optical system 28. In this case a separate window 36 for transmitting thermal infrared radiation is needed in the airway adapter 8 opposite the luminophore 13. The material or thickness of the body 14 is not so critical, because the thermal radiation from the luminophore is measured directly across the airway adapter 8 without being conducted through the body 14 to measure the temperature of the luminophore.

The infrared radiation detector is advantageously a thermopile detector. With a thermopile detector, an optical chopper is not needed. Furthermore, integrated components for infrared thermometry are readily available. An example of such a component is the One Channel Thermopile Detector, TS1x80B-A-D0.48 manufactured by Micro Hybrid Electronic, Hermsdorf, Germany. If necessary, the component may also comprise a lens or a reflector for collecting radiation to the detector. Other types of infrared radiation detectors, such as pyroelectric detectors or bolometer detectors can naturally be used.

The radiation power (Pdet) falling to the infrared detector depends on the temperature of the surface filling the field of view of the detector (Tlp) and the reference temperature (Tref) of the infrared detector, as well as the radiant properties of the surfaces. The equation can be derived from the from the Stefan-Bolzmann's law:

Pdet=R*(Tlp̂4−Tref̂4),

where R is a constant depending on radiant properties of the surface whose temperature is measured, the optical filter used and the optical system directing radiation from the emitting surface to the detector.

For thermopile detectors:

Vdet=S*Pdet=>Pdet=Vdet/S,

where S is the sensitivity of the thermopile detector

Thus, the temperature of the luminophore, Tlump:

Tlp=[(Vdet/S+K*Tref̂4)/K]̂(1/4)

The temperature of the luminophore is needed to correct the measurement results of the oxygen concentration, because the temperature of the luminophore is varying having an influence on the measurement results of oxygen. So it is important to know the temperature of the luminophore and correct the oxygen concentration measurement results accordingly.

The gas analyzer 7 may also comprise a processing unit 27 receiving a signal indicative of an oxygen concentration from the oxygen detector, and receiving a signal indicative of a temperature of the luminophore 13 and receiving a signal indicative of the temperature of the infrared detector. The processing unit may also determine the oxygen concentration of the respiratory gas based on the signal indicative of an oxygen concentration, the signal indicative of a temperature of the luminophore 13 and the signal indicative of the temperature of the infrared detector. The processing unit for the infrared temperature measurement can be such that it calculates the temperature of the luminophore. The necessary processing may be performed in the same processing unit that also calculates the concentrations of oxygen and other gases measured by the gas sensor. Instead of one common processing unit there may be for example two different processing units, one for the infrared thermometry unit 25 and another for oxygen concentration measurement, which may be common to the infrared gas analysis functions. The signal conditioning electronics in the gas sensor may only perform suitable conditioning of electric signals obtained from the IR-detector and reference temperature sensor so that these signals can be transmitted to a processing unit located away from the gas sensor.

The emitter 12 for exciting the luminophore 13 and the oxygen detector 16 for detecting the luminescent radiation are located in a gas sensor 23 which is part of the gas analyzer 7 and may not be disposable. The gas sensor 23 may be mountable on the airway adapter 8. Optically, the construction can be made in a number of ways, 5 of which are shown in FIGS. 2, 3, 4, 5 and 6 where exciting radiation rays 19 such as light rays from the emitter 12 enter the body 14 made of transparent material through one end and passes through the body to the luminophore 13. At some instance the radiation rays 19 will excite the luminophore 13. The consequently emitted luminescence is emitted in all directions and part of the luminescent radiation 24 will enter detector 16. An optical arrangement such as a lens or mirror can be used for collecting the emitted radiation to detector 16.

Oxygen in contact with the luminophore 13 will quench the luminescence and a signal related to the concentration of oxygen can be calculated and displayed for instance in the patient monitor 10. This is done using well-known principles and applying the Stern-Volmer relationship

I₀/I=1+K(T)·C(O₂),

where I₀ is the luminescence intensity in absence of oxygen, I is the measured intensity at concentration C(O₂) of oxygen. The constant K(T) is the Stern-Volmer constant at luminophore temperature T. This equation could also be written as

τ₀/τ=1+K(T)·C(O₂),

where τ₀ is the luminescence decay time in absence of oxygen and τ is the measured decay time at concentration C(O₂) of oxygen. The method is well known and described in detail e.g. in the document Kolle, C. et al.: Fast optochemical sensor for continuous monitoring of oxygen in breath-gas analysis, Sensors and Actuators B 38-39 (1997) 141-149.

Although Kolle, C. et al. do not explicitly present the formula for the temperature dependence of the Stern-Volmer constant K(T), they keep the temperature of their sensor at known level and use an additional microchip thermistor for obtaining a useful estimate for the temperature of the fluorophore when it is changed by the gas flowing by. They also present a graph that demonstrates the need for knowing the instantaneous temperature of the fluorophore even if the sensor temperature is stabilized. The thermal stabilization and measurement significantly add to the bulkiness, complexity and power consumption of the sensor, which is avoided in embodiments explained hereinbefore.

The written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

I claim:

1. A gas sensor comprising:
an emitter for emitting radiation to a body at least partly coated with a luminophore emitting luminescent radiation indicative of an oxygen concentration when in contact with a respiratory gas;
a filter for transmitting the luminescent radiation emitted by said luminophore;
an oxygen detector for receiving the luminescent radiation transmitted by said filter; and
an infrared thermometry unit for receiving a thermal radiation indicative of a temperature of said luminophore.

2. The gas sensor according to claim 1, wherein said infrared thermometry unit comprises an infrared detector for receiving a thermal radiation and a temperature sensor for measuring the temperature of the infrared detector.

3. The gas sensor according to claim 2, wherein said oxygen detector is adapted to provide a signal based on the received luminescent radiation indicative of an oxygen concentration and that said infrared thermometry unit is adapted to provide a signal based on the received thermal radiation indicative of a temperature of said luminophore and that said temperature sensor is adapted to provide a signal based on the temperature of the infrared detector.

4. The gas sensor according to claim 3 further comprising a processing unit for receiving a signal indicative of an oxygen concentration and for receiving a signal indicative of a temperature of said luminophore and receiving a signal indicative of the temperature of said infrared detector.

5. The gas sensor according to claim 4, wherein said processing unit is adapted to determine the oxygen concentration of the respiratory gas based on said signal indicative of an oxygen concentration, said signal indicative of a temperature of said luminophore and said signal indicative of the temperature of said infrared detector.

6. The gas sensor according to claim 2, wherein said infrared thermometry unit further comprises an optical system to limit the field of view of said infrared detector to a suitable portion of said luminophore and collecting radiation thermally emitted from that portion to said infrared detector.

7. The gas sensor according to claim 6, wherein said optical system comprises an aperture for limiting the field of view of the infrared detector and an optical filter for passing a suitable range of IR wavelengths and one of a mirror for reflecting the thermal radiation, a reflector for reflecting the thermal radiation and a lens for collecting and focusing the thermal radiation.

8. The gas sensor according to claim 1 further comprising an infrared source for emitting radiation through the respiratory gas and at least one gas detector for providing a signal indicative of at least one respiratory gas other than oxygen.

9. A gas analyzer for measuring oxygen concentration of a respiratory gas comprising:
an emitter for emitting radiation;
an airway adapter having a flow channel carrying respiratory gas including oxygen;
a body at least partly coated with a luminophore excited by the radiation emitted by said emitter, said luminophore being in contact with said respiratory gas and emitting luminescent radiation;
a filter for transmitting the luminescent radiation emitted by said luminophore;
an oxygen detector for receiving the luminescent radiation transmitted by said filter; and
an infrared thermometry unit for receiving a thermal radiation from said luminophore.

10. The gas analyzer according to claim 9, wherein said infrared thermometry unit comprises an infrared detector for receiving a thermal radiation and a temperature sensor for measuring the temperature of the infrared detector.

11. The gas analyzer according to claim 10, wherein said oxygen detector is adapted to provide a signal based on the received luminescent radiation indicative of an oxygen concentration and that said thermometry unit is adapted to provide a signal based on the received thermal radiation signal indicative of a temperature of said luminophore and that said temperature sensor is adapted to provide a signal based on the temperature of said infrared detector.

12. The gas analyzer according to claim 11 further comprising a processing unit for receiving a signal indicative of an oxygen concentration and for receiving a signal indicative of a temperature of said luminophore and for receiving a signal based on the temperature of said infrared detector.

13. The gas analyzer according to claim 12, wherein said processing unit is adapted to determine the oxygen concentration of the respiratory gas based on said signal indicative of an oxygen concentration, said signal indicative of a temperature of said luminophore and said signal based on the temperature of said infrared detector.

14. The gas analyzer according to claim 9, wherein said body is a window.

15. The gas analyzer according to claim 9 further comprising an infrared source for emitting radiation through the respiratory gas and at least one gas detector for providing a signal indicative of at least one respiratory gas other than oxygen.

16. The gas analyzer according to claim 10, wherein the infrared detector is detached from the body.

17. A method for measuring oxygen concentration of a respiratory gas comprising:
emitting a radiation to a body coated at least partly with a luminophore which luminophore is adapted to emit luminescent radiation indicative of an oxygen concentration when in contact with the respiratory gas;
filtering the radiation to transmit the luminescent radiation;
detecting the transmitted luminescent radiation; and
receiving a thermal radiation from said luminophore indicative of a temperature of said luminophore.

18. The method according to claim 17 further comprising measuring a temperature of the detecting phase.

19. The method according to claim 18 further comprising providing a signal based on said detecting indicative of the oxygen concentration, and providing a signal based on received thermal radiation and providing a signal based on the temperature of the detecting phase and determining based on these signals the oxygen concentration of the respiratory gas.

20. The method according to claim 15, wherein said receiving a thermal radiation is adapted to be done at a distance from said luminophore.
